SEQUENCE LISTING (127 sequences)

SEQ ID NO: 1 - protein, 25 aa, synthetic construct
  VARIANT 2: note = X can be any amino acid
  VARIANT 3: note = X can be any amino acid
  VARIANT 4: note = X can be any amino acid
  VARIANT 5: note = X can be any amino acid
  VARIANT 7: note = X can be any amino acid
  VARIANT 8: note = X can be any amino acid
  VARIANT 9: note = X can be any amino acid
  VARIANT 10: note = X can be any amino acid
  VARIANT 11: note = X can be any amino acid
  VARIANT 12: note = X can be any amino acid
  VARIANT 13: note = X can be any amino acid
  VARIANT 14: note = X can be any amino acid
  VARIANT 15: note = X can be any amino acid
  VARIANT 16: note = X can be any amino acid
  VARIANT 17: note = X can be any amino acid
  VARIANT 18: note = X can be any amino acid
  VARIANT 20: note = X can be any amino acid
  VARIANT 21: note = X can be any amino acid
  VARIANT 22: note = X can be any amino acid
  VARIANT 23: note = X can be any amino acid
  VARIANT 24: note = X can be any amino acid
CXXXXCXXXXXXXXXXXXHXXXXXH

SEQ ID NO: 2 - protein, 5 aa, synthetic construct
TGEKP

SEQ ID NO: 3 - protein, 197 aa, synthetic construct
  VARIANT 34: note = X can be any amino acid
  VARIANT 35: note = X can be any amino acid
  VARIANT 36: note = X can be any amino acid
  VARIANT 37: note = X can be any amino acid
  VARIANT 38: note = X can be any amino acid
  VARIANT 39: note = X can be any amino acid
  VARIANT 40: note = X can be any amino acid
  VARIANT 62: note = X can be any amino acid
  VARIANT 63: note = X can be any amino acid
  VARIANT 64: note = X can be any amino acid
  VARIANT 65: note = X can be any amino acid
  VARIANT 66: note = X can be any amino acid
  VARIANT 67: note = X can be any amino acid
  VARIANT 68: note = X can be any amino acid
  VARIANT 91: note = X can be any amino acid
  VARIANT 92: note = X can be any amino acid
  VARIANT 93: note = X can be any amino acid
  VARIANT 94: note = X can be any amino acid
  VARIANT 95: note = X can be any amino acid
  VARIANT 96: note = X can be any amino acid
  VARIANT 97: note = X can be any amino acid
  VARIANT 119: note = X can be any amino acid
  VARIANT 120: note = X can be any amino acid
  VARIANT 121: note = X can be any amino acid
  VARIANT 122: note = X can be any amino acid
  VARIANT 123: note = X can be any amino acid
  VARIANT 124: note = X can be any amino acid
  VARIANT 125: note = X can be any amino acid
  VARIANT 148: note = X can be any amino acid
  VARIANT 149: note = X can be any amino acid
  VARIANT 150: note = X can be any amino acid
  VARIANT 151: note = X can be any amino acid
  VARIANT 152: note = X can be any amino acid
  VARIANT 153: note = X can be any amino acid
  VARIANT 154: note = X can be any amino acid
  VARIANT 176: note = X can be any amino acid
  VARIANT 177: note = X can be any amino acid
  VARIANT 178: note = X can be any amino acid
  VARIANT 179: note = X can be any amino acid
  VARIANT 180: note = X can be any amino acid
  VARIANT 181: note = X can be any amino acid
  VARIANT 182: note = X can be any amino acid
MAPKKKRKVGIHGVPAAMAERPFQCRICMRNFSXXXXXXXHIRTHTGEKPFACDICGRKFAXXXXXXXHTKIHTGSQKPFQCRICMRNFSXXXXXXXHIRTHTGEKPFACDICGRKFAXXXXXXXHTKIHTGSQKPFQCRICMRNFSXXXXXXXHIRTHTGEKPFACDICGRKFAXXXXXXXHTKIHLRQKDAARGS

SEQ ID NO: 4 - protein, 197 aa, synthetic construct; identical to SEQ ID NO: 3
  VARIANT 34: note = X can be any amino acid
  VARIANT 35: note = X can be any amino acid
  VARIANT 36: note = X can be any amino acid
  VARIANT 37: note = X can be any amino acid
  VARIANT 38: note = X can be any amino acid
  VARIANT 39: note = X can be any amino acid
  VARIANT 40: note = X can be any amino acid
  VARIANT 62: note = X can be any amino acid
  VARIANT 63: note = X can be any amino acid
  VARIANT 64: note = X can be any amino acid
  VARIANT 65: note = X can be any amino acid
  VARIANT 66: note = X can be any amino acid
  VARIANT 67: note = X can be any amino acid
  VARIANT 68: note = X can be any amino acid
  VARIANT 91: note = X can be any amino acid
  VARIANT 92: note = X can be any amino acid
  VARIANT 93: note = X can be any amino acid
  VARIANT 94: note = X can be any amino acid
  VARIANT 95: note = X can be any amino acid
  VARIANT 96: note = X can be any amino acid
  VARIANT 97: note = X can be any amino acid
  VARIANT 119: note = X can be any amino acid
  VARIANT 120: note = X can be any amino acid
  VARIANT 121: note = X can be any amino acid
  VARIANT 122: note = X can be any amino acid
  VARIANT 123: note = X can be any amino acid
  VARIANT 124: note = X can be any amino acid
  VARIANT 125: note = X can be any amino acid
  VARIANT 148: note = X can be any amino acid
  VARIANT 149: note = X can be any amino acid
  VARIANT 150: note = X can be any amino acid
  VARIANT 151: note = X can be any amino acid
  VARIANT 152: note = X can be any amino acid
  VARIANT 153: note = X can be any amino acid
  VARIANT 154: note = X can be any amino acid
  VARIANT 176: note = X can be any amino acid
  VARIANT 177: note = X can be any amino acid
  VARIANT 178: note = X can be any amino acid
  VARIANT 179: note = X can be any amino acid
  VARIANT 180: note = X can be any amino acid
  VARIANT 181: note = X can be any amino acid
  VARIANT 182: note = X can be any amino acid

SEQ ID NO: 5 - protein, 191 aa, synthetic construct
  VARIANT 34: note = X can be any amino acid
  VARIANT 35: note = X can be any amino acid
  VARIANT 36: note = X can be any amino acid
  VARIANT 37: note = X can be any amino acid
  VARIANT 38: note = X can be any amino acid
  VARIANT 39: note = X can be any amino acid
  VARIANT 40: note = X can be any amino acid
  VARIANT 62: note = X can be any amino acid
  VARIANT 63: note = X can be any amino acid
  VARIANT 64: note = X can be any amino acid
  VARIANT 65: note = X can be any amino acid
  VARIANT 66: note = X can be any amino acid
  VARIANT 67: note = X can be any amino acid
  VARIANT 68: note = X can be any amino acid
  VARIANT 90: note = X can be any amino acid
  VARIANT 91: note = X can be any amino acid
  VARIANT 92: note = X can be any amino acid
  VARIANT 93: note = X can be any amino acid
  VARIANT 94: note = X can be any amino acid
  VARIANT 95: note = X can be any amino acid
  VARIANT 96: note = X can be any amino acid
  VARIANT 118: note = X can be any amino acid
  VARIANT 119: note = X can be any amino acid
  VARIANT 120: note = X can be any amino acid
  VARIANT 121: note = X can be any amino acid
  VARIANT 122: note = X can be any amino acid
  VARIANT 123: note = X can be any amino acid
  VARIANT 124: note = X can be any amino acid
  VARIANT 146: note = X can be any amino acid
  VARIANT 147: note = X can be any amino acid
  VARIANT 148: note = X can be any amino acid
  VARIANT 149: note = X can be any amino acid
  VARIANT 150: note = X can be any amino acid
  VARIANT 151: note = X can be any amino acid
  VARIANT 152: note = X can be any amino acid
  VARIANT 174: note = X can be any amino acid
  VARIANT 175: note = X can be any amino acid
  VARIANT 176: note = X can be any amino acid
  VARIANT 177: note = X can be any amino acid
  VARIANT 178: note = X can be any amino acid
  VARIANT 179: note = X can be any amino acid
  VARIANT 180: note = X can be any amino acid
ISEFGSSSSVAQAALEPGEKPYACPECGKSFSRXXXXXXXHQRTHTGEKPYKCPECGKSFSXXXXXXXHQRTHTGEKPYKCPECGKSFSXXXXXXXHQRTHTGEKPYKCPECGKSFSXXXXXXXHQRTHTGEKPYKCPECGKSFSXXXXXXXHQRTHTGEKPYKCPECGKSFSXXXXXXXHQRTHTGKKTS

SEQ ID NO: 6 - protein, 196 aa, synthetic construct
  VARIANT 23: note = X can be any amino acid
  VARIANT 24: note = X can be any amino acid
  VARIANT 25: note = X can be any amino acid
  VARIANT 26: note = X can be any amino acid
  VARIANT 27: note = X can be any amino acid
  VARIANT 28: note = X can be any amino acid
  VARIANT 29: note = X can be any amino acid
  VARIANT 53: note = X can be any amino acid
  VARIANT 54: note = X can be any amino acid
  VARIANT 55: note = X can be any amino acid
  VARIANT 56: note = X can be any amino acid
  VARIANT 57: note = X can be any amino acid
  VARIANT 58: note = X can be any amino acid
  VARIANT 59: note = X can be any amino acid
  VARIANT 81: note = X can be any amino acid
  VARIANT 82: note = X can be any amino acid
  VARIANT 83: note = X can be any amino acid
  VARIANT 84: note = X can be any amino acid
  VARIANT 85: note = X can be any amino acid
  VARIANT 86: note = X can be any amino acid
  VARIANT 87: note = X can be any amino acid
  VARIANT 120: note = X can be any amino acid
  VARIANT 121: note = X can be any amino acid
  VARIANT 122: note = X can be any amino acid
  VARIANT 123: note = X can be any amino acid
  VARIANT 124: note = X can be any amino acid
  VARIANT 125: note = X can be any amino acid
  VARIANT 126: note = X can be any amino acid
  VARIANT 150: note = X can be any amino acid
  VARIANT 151: note = X can be any amino acid
  VARIANT 152: note = X can be any amino acid
  VARIANT 153: note = X can be any amino acid
  VARIANT 154: note = X can be any amino acid
  VARIANT 155: note = X can be any amino acid
  VARIANT 156: note = X can be any amino acid
  VARIANT 178: note = X can be any amino acid
  VARIANT 179: note = X can be any amino acid
  VARIANT 180: note = X can be any amino acid
  VARIANT 181: note = X can be any amino acid
  VARIANT 182: note = X can be any amino acid
  VARIANT 183: note = X can be any amino acid
  VARIANT 184: note = X can be any amino acid
VPIPGKKKQHICHIQGCGKVYGXXXXXXXHLRWHTGERPFMCTWSYCGKRFTXXXXXXXHKRTHTGEKKFACPECPKRFMXXXXXXXHIKTHQNKKDGGGSGKKKQHICHIQGCGKVYGXXXXXXXHLRWHTGERPFMCTWSYCGKRFTXXXXXXXHKRTHTGEKKFACPECPKRFMXXXXXXXHIKTHQNKKGGS

SEQ ID NO: 7 - protein, 7 aa, synthetic construct
RSDSLSR

SEQ ID NO: 8 - protein, 7 aa, synthetic construct
RNASLAT

SEQ ID NO: 9 - protein, 7 aa, synthetic construct
DRSDLSR

SEQ ID NO: 10 - protein, 7 aa, synthetic construct
DRSDLSR

SEQ ID NO: 11 - protein, 7 aa, synthetic construct
DRSALSR

SEQ ID NO: 12 - protein, 7 aa, synthetic construct
QSGTLAR

SEQ ID NO: 13 - protein, 7 aa, synthetic construct
RSSDLTE

SEQ ID NO: 14 - protein, 7 aa, synthetic construct
RSADLSR

SEQ ID NO: 15 - protein, 7 aa, synthetic construct
QSSDLSR

SEQ ID NO: 16 - protein, 7 aa, synthetic construct
HRSTLSR

SEQ ID NO: 17 - protein, 7 aa, synthetic construct
QSSDLSR

SEQ ID NO: 18 - protein, 7 aa, synthetic construct
QSSTLSR

SEQ ID NO: 19 - protein, 7 aa, synthetic construct
QSSDLSR

SEQ ID NO: 20 - protein, 7 aa, synthetic construct
QSGHLSR

SEQ ID NO: 21 - protein, 7 aa, synthetic construct
RSDVLSE

SEQ ID NO: 22 - protein, 7 aa, synthetic construct
RNQHRKT

SEQ ID NO: 23 - protein, 7 aa, synthetic construct
YPKDLSK

SEQ ID NO: 24 - protein, 7 aa, synthetic construct
QSGNLAR

SEQ ID NO: 25 - protein, 7 aa, synthetic construct
RSDSLTR

SEQ ID NO: 26 - protein, 7 aa, synthetic construct
QSGDLTR

SEQ ID NO: 27 - protein, 7 aa, synthetic construct
QSGDLTR

SEQ ID NO: 28 - protein, 7 aa, synthetic construct
QSGDLTR

SEQ ID NO: 29 - protein, 7 aa, synthetic construct
QSGDLTR

SEQ ID NO: 30 - protein, 7 aa, synthetic construct
QSGDLTR

SEQ ID NO: 31 - protein, 7 aa, synthetic construct
ERGTLTR

SEQ ID NO: 32 - protein, 7 aa, synthetic construct
RSDALAR

SEQ ID NO: 33 - protein, 7 aa, synthetic construct
DRDSLTR

SEQ ID NO: 34 - protein, 7 aa, synthetic construct
DRSNLAR

SEQ ID NO: 35 - protein, 7 aa, synthetic construct
RSDNLAR

SEQ ID NO: 36 - protein, 7 aa, synthetic construct
RSDNLTR

SEQ ID NO: 37 - protein, 7 aa, synthetic construct
QSGSLTR

SEQ ID NO: 38 - protein, 7 aa, synthetic construct
QSGNLAR

SEQ ID NO: 39 - protein, 7 aa, synthetic construct
ERRDLSR

SEQ ID NO: 40 - protein, 7 aa, synthetic construct
RSDALPM

SEQ ID NO: 41 - protein, 7 aa, synthetic construct
DRSDLSR

SEQ ID NO: 42 - protein, 7 aa, synthetic construct
DRSNLTR

SEQ ID NO: 43 - protein, 7 aa, synthetic construct
RSDNLSR

SEQ ID NO: 44 - protein, 7 aa, synthetic construct
RSADLSR

SEQ ID NO: 45 - protein, 7 aa, synthetic construct
QSGDLTR

SEQ ID NO: 46 - protein, 7 aa, synthetic construct
RRDHLTT

SEQ ID NO: 47 - protein, 7 aa, synthetic construct
QSDVLSR

SEQ ID NO: 48 - protein, 7 aa, synthetic construct
QSATLSK

SEQ ID NO: 49 - protein, 7 aa, synthetic construct
RSDNLSR

SEQ ID NO: 50 - protein, 7 aa, synthetic construct
DRGTLAR

SEQ ID NO: 51 - protein, 7 aa, synthetic construct
DRSNLSR

SEQ ID NO: 52 - protein, 7 aa, synthetic construct
QSSNLAR

SEQ ID NO: 53 - protein, 7 aa, synthetic construct
RSDSLSR

SEQ ID NO: 54 - protein, 7 aa, synthetic construct
DRSNRKT

SEQ ID NO: 55 - protein, 7 aa, synthetic construct
QSGTRKT

SEQ ID NO: 56 - protein, 7 aa, synthetic construct
RSDNLAR

SEQ ID NO: 57 - protein, 7 aa, synthetic construct
DRSDLTR

SEQ ID NO: 58 - protein, 7 aa, synthetic construct
DRSNLAK

SEQ ID NO: 59 - protein, 7 aa, synthetic construct
QSSHLAR

SEQ ID NO: 60 - protein, 7 aa, synthetic construct
RSDALAR

SEQ ID NO: 61 - protein, 7 aa, synthetic construct
QSSNLVR

SEQ ID NO: 62 - protein, 7 aa, synthetic construct
RSDKLSR

SEQ ID NO: 63 - protein, 7 aa, synthetic construct
QSSALSR

SEQ ID NO: 64 - protein, 7 aa, synthetic construct
RSDNLAR

SEQ ID NO: 65 - protein, 7 aa, synthetic construct
DRSDLSR

SEQ ID NO: 66 - protein, 7 aa, synthetic construct
DRSNLSR

SEQ ID NO: 67 - protein, 7 aa, synthetic construct
ERGTLAR

SEQ ID NO: 68 - protein, 7 aa, synthetic construct
QSSDLRR

SEQ ID NO: 69 - protein, 7 aa, synthetic construct
QSSDLSR

SEQ ID NO: 70 - protein, 7 aa, synthetic construct
RSDHLRR

SEQ ID NO: 71 - protein, 7 aa, synthetic construct
RSDHLSR

SEQ ID NO: 72 - protein, 7 aa, synthetic construct
RSDDRKT

SEQ ID NO: 73 - protein, 7 aa, synthetic construct
RSDNLAR

SEQ ID NO: 74 - protein, 7 aa, synthetic construct
RSDNLAR

SEQ ID NO: 75 - protein, 7 aa, synthetic construct
RSAHLSR

SEQ ID NO: 76 - protein, 7 aa, synthetic construct
RSDHLSR

SEQ ID NO: 77 - protein, 7 aa, synthetic construct
RSDHLSN

SEQ ID NO: 78 - protein, 7 aa, synthetic construct
RSDHRKT

SEQ ID NO: 79 - protein, 7 aa, synthetic construct
DRSHLSR

SEQ ID NO: 80 - protein, 7 aa, synthetic construct
RSDHLSR

SEQ ID NO: 81 - protein, 7 aa, synthetic construct
RSDALAR

SEQ ID NO: 82 - protein, 7 aa, synthetic construct
RSDHLSR

SEQ ID NO: 83 - protein, 7 aa, synthetic construct
RSDNLSA

SEQ ID NO: 84 - protein, 7 aa, synthetic construct
RKHDRIT

SEQ ID NO: 85 - protein, 7 aa, synthetic construct
RSDDLAR

SEQ ID NO: 86 - protein, 7 aa, synthetic construct
RSDHLSR

SEQ ID NO: 87 - protein, 7 aa, synthetic construct
RSDNLAR

SEQ ID NO: 88 - protein, 7 aa, synthetic construct
RSDHLAR

SEQ ID NO: 89 - protein, 7 aa, synthetic construct
RSDNLAR

SEQ ID NO: 90 - protein, 7 aa, synthetic construct
QSGHLSR

SEQ ID NO: 91 - protein, 7 aa, synthetic construct
RSDHLSA

SEQ ID NO: 92 - protein, 7 aa, synthetic construct
RSAHLTN

SEQ ID NO: 93 - protein, 7 aa, synthetic construct
RSDHLSR

SEQ ID NO: 94 - protein, 7 aa, synthetic construct
RSDALAR

SEQ ID NO: 95 - protein, 7 aa, synthetic construct
QSGHLAR

SEQ ID NO: 96 - protein, 7 aa, synthetic construct
RSDHLTT

SEQ ID NO: 97 - protein, 7 aa, synthetic construct
QSSDLRR

SEQ ID NO: 98 - protein, 7 aa, synthetic construct
RSDHLSR

SEQ ID NO: 99 - protein, 7 aa, synthetic construct
RSDSLLR

SEQ ID NO: 100 - protein, 7 aa, synthetic construct
QSGHLTR

SEQ ID NO: 101 - protein, 7 aa, synthetic construct
RSDHLSR

SEQ ID NO: 102 - protein, 7 aa, synthetic construct
RSDHLSR

SEQ ID NO: 103 - protein, 7 aa, synthetic construct
QSSDLTR

SEQ ID NO: 104 - protein, 7 aa, synthetic construct
DRKSLAR

SEQ ID NO: 105 - protein, 7 aa, synthetic construct
QSGDLTR

SEQ ID NO: 106 - protein, 7 aa, synthetic construct
QSGHLSR

SEQ ID NO: 107 - protein, 7 aa, synthetic construct
RSDHLSR

SEQ ID NO: 108 - protein, 7 aa, synthetic construct
RSDALAR

SEQ ID NO: 109 - protein, 7 aa, synthetic construct
RSDDLTR

SEQ ID NO: 110 - protein, 7 aa, synthetic construct
RSDNLAR

SEQ ID NO: 111 - protein, 7 aa, synthetic construct
DRSHLSR

SEQ ID NO: 112 - protein, 7 aa, synthetic construct
RSDHLSR

SEQ ID NO: 113 - protein, 7 aa, synthetic construct
RSDHLSR

SEQ ID NO: 114 - protein, 7 aa, synthetic construct
RNDHRKR

SEQ ID NO: 115 - protein, 7 aa, synthetic construct
RSDHLSR

SEQ ID NO: 116 - protein, 7 aa, synthetic construct
RSADLRR

SEQ ID NO: 117 - protein, 7 aa, synthetic construct
RSDNLAR

SEQ ID NO: 118 - protein, 7 aa, synthetic construct
DRSHLAR

SEQ ID NO: 119 - protein, 7 aa, synthetic construct
RSDHLSR

SEQ ID NO: 120 - protein, 7 aa, synthetic construct
RSDHRKT

SEQ ID NO: 121 - protein, 15 aa, synthetic construct
RNFSRSDHLSTHIRT

SEQ ID NO: 122 - protein, 15 aa, synthetic construct
RKFARSDNLTRHTKI

SEQ ID NO: 123 - protein, 15 aa, synthetic construct
RNFSQNANRKTHIRT

SEQ ID NO: 124 - protein, 15 aa, synthetic construct
  VARIANT 5: note = X can be any amino acid
  VARIANT 6: note = X can be any amino acid
  VARIANT 7: note = X can be any amino acid
  VARIANT 8: note = X can be any amino acid
  VARIANT 9: note = X can be any amino acid
  VARIANT 10: note = X can be any amino acid
  VARIANT 11: note = X can be any amino acid
RNFSXXXXXXXHIRT

SEQ ID NO: 125 - protein, 21 aa, synthetic construct
RSDALRQTSGELVRRGDALTS

SEQ ID NO: 126 - protein, 23 aa, synthetic construct
RSDALRQZTSGELVRZRGDALTS

SEQ ID NO: 127 - DNA, 25 bp, synthetic construct
  source 1..25: mol_type = other DNA
ttggttggctggctagctatctatg

(19) United States
(12) Patent Application Publication
(10) Pub. No.: US 2026/0196298 A1
(43) Pub. Date: Jul. 9, 2026

(54) ZINC FINGER ENGINEERING

(71) Applicant: The Regents of the University of California, Oakland, CA (US)

(72) Inventors: Prashant Mali, La Jolla, CA (US); Michael Tong, La Jolla, CA (US)

(21) Appl. No.: 19/129,810

(22) PCT Filed: Nov. 15, 2023
(86) PCT No.: PCT/US2023/079851

§ 371 Date: May 14, 2025

Related U.S. Application Data
(60) Provisional application No. 63/426,034, filed on Nov. 16, 2022.

(51) Int. Cl. G16B 20/30; G06N 3/0442; G06N 3/045; G16B 30/00; G16B 40/20

(52) U.S. Cl. CPC G16B 20/30; G06N 3/0442; G06N 3/045; G16B 30/00; G16B 40/20

(57) ABSTRACT

Herein is described a training method, comprising: receiving data representing an amino acid sequence and a DNA sequence; based on the received data, generating a set of pairs of corresponding zinc finger array sequences of the amino acid sequence and DNA sub-sequences of the DNA sequence, wherein a DNA sub-sequence comprises a binding site for a corresponding zinc finger array sequence; generating training data based at least in part the set of pairs of corresponding zinc finger array sequences and DNA subsequences; training, using the training data, a machine learning model to predict, from an input DNA sequence comprising a set of input DNA sub-sequences, a set of output zinc finger array sequences, an output zinc finger array sequence of the set of output zinc finger array sequences corresponding to an input DNA sub-sequence of the set of input DNA sub-sequences.

### CROSS-REFERENCE

This application claims priority to U.S. Provisional Application No. 63/426,034, entitled “Zinc Finger Engineering,” filed Nov. 16, 2022, which is entirely herein incorporated by reference.

### BACKGROUND

The C₂H₂ class of zinc finger protein structural motifs (“zinc fingers”), characterized by a -Cys-(X)₂₋₄-Cys-(X)₁₂-His-(X)₃₋₅-His-motif where X represents any amino acid (aa), hold advantages over other engineered DNA targeting proteins, such as low immunogenicity, direct delivery capability, and small size. Neighboring C₂H₂ zinc fingers have also been shown to affect DNA binding specificity, demonstrating a need to capture context between zinc finger modules. Modular generation of these zinc finger arrays (ZFAs) from established lookup tables has been shown to be both resource-intensive and low-yield. Studies have characterized the modularity of zinc fingers. But the vast majority of zinc finger arrays have been exclusively characterized only by three-zinc finger constructs, with few studies characterizing six-zinc finger constructs. Thus, there is a need for a robust and rapid pipeline to generate high-affinity high-specificity six finger polydactyl zinc finger arrays.

### SUMMARY

In some example embodiments, there may be provided a method including receiving data representing an amino acid sequence and a DNA sequence; based on the received data, generating a set of pairs of corresponding zinc finger array sequences of the amino acid sequence and DNA sub-sequences of the DNA sequence, wherein a DNA sub-sequence comprises a binding site for a corresponding zinc finger array sequence; generating training data based at least in part the set of pairs of corresponding zinc finger array sequences and DNA subsequences; training, using the training data, a machine learning model to predict, from an input DNA sequence comprising a set of input DNA sub-sequences, a set of output zinc finger array sequences, an output zinc finger array sequence of the set of output zinc finger array sequences corresponding to an input DNA sub-sequence of the set of input DNA sub-sequences. In some variations, one or more of the features disclosed herein including the following features can optionally be included in any feasible combination. A position of the DNA binding amino acid is −1, 2, 3, or 6. A first DNA sub-sequence of the DNA sub-sequences is a 5-mer and a second DNA sub-sequence of the DNA sub-sequences is a 5-mer. The first DNA sub-sequence is a first set of consecutive bases of the DNA sequence and the second DNA sub sequence is a second set of consecutive bases of the DNA sequence. The second set is determined at least in part by shifting the first set. The shifting is by one base of the DNA sequence. The shifting is rightward. The first zinc finger sub-sequence is a 3-mer and the second zinc finger sub-sequence is a 3-mer. The first zinc finger sub-sequence corresponds to a first set of amino acids of the zinc finger array sequence. The second zinc finger sub-sequence corresponds to a second set of amino acids of the zinc finger array sequence. The second zinc finger sub-sequence is determined by shifting the first zinc finger sub-sequence. The shifting is rightward. The shifting is by two amino acids of the zinc finger array sequence. The trained machine learning model comprises a neural network, the neural network is a bidirectional neural network. The bidirectional neural network comprises a gated recurrent unit (GRU).

In some example embodiments, there may be provided a method including receiving input data comprising an input DNA sequence; and predicting, using a trained machine learning model, a set of output zinc finger array sequences, an output zinc finger array sequence corresponding to a DNA sub-sequence of the input DNA sequence.

In some variations, one or more of the features disclosed herein including the following features can be optionally included in any feasible combination. The DNA sub-sequence comprises a binding site for the corresponding output zinc finger array sequence. The trained machine learning model comprises a neural network. The neural network is a bidirectional neural network. The bidirectional neural network comprises a gated recurrent unit (GRU). The method may further comprise inserting the set of output zinc finger array sequences into a zinc finger backbone. The method may further comprise generating a confidence metric at least in part by processing the zinc finger backbone comprising the set of output zinc finger array sequences with a second machine learning model. The second machine learning model comprises a support vector machine model, a zinc finger array sequence comprises a 7-mer amino acid sequence. A DNA sub-sequence comprises a 3-mer DNA base sequence.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 illustrates a system configured to generate and validate a zinc finger protein sequence, in accordance with some embodiments, in accordance with some embodiments;

FIG. 2 illustrates a k-mer embedding method, in accordance with some embodiments;

FIG. 3 illustrates a process flow diagram for training a machine learning unit, in accordance with some embodiments;

FIG. 4 illustrates a process flow diagram for using a machine learning unit to generate a set of output zinc finger array sequences, in accordance with some embodiments;

FIG. 5 illustrates instanced repression and inheritable silencing by zinc finger arrays bound to KRAB and KRAB+3A3L;

FIG. 6 illustrates zinc finger array and domain DNA binding conformations, in accordance with some embodiments;

FIG. 7 illustrates successful six finger ZFA-KRAB Fusions for >50% hPCSK9 repression;

FIG. 8 illustrates successful six finger ZFA-KRAB fusions for >50% CXCR4 repression; and

FIG. 9 illustrates successful six finger ZFA-KRAB fusions for >100% ASCL1 activation.

### DETAILED DESCRIPTION

A sequence of zinc finger arrays (or “modules”) can be generated using machine learning models implementing natural language processing techniques. First, amino acid sequence data (e.g., natural zinc fingers) and DNA sequence data (e.g., zinc finger targets) is collected. Then, “word pairs” of zinc finger arrays (e.g., DNA contact regions comprising seven amino acids) from the amino acid sequence data and corresponding target sites (e.g., comprising three DNA base pairs) are extracted from the collected data. These word pairs may be arranged into “sentences” (comprising, for example, three to six adjacent zinc finger arrays). These “sentences” are used to train a machine learning model to generate a set of zinc finger arrays given a sequence of DNA base pairs. The trained machine learning model can then generate a set of zinc finger arrays corresponding to a provided DNA sequence. The machine learning model may be a recurrent neural network, such as a bidirectional neural network (BiRNN) with gated recurrent units (GRUs).

In some cases, a k-mer embedding scheme is used to pre-process the zinc finger array data and DNA sequence data. This pre-processing includes generating, from a padded seven amino acid zinc finger array, a set of three overlapping 3-mer tokens. The 3-mers, when arranged, form a version of the zinc finger array with key DNA binding amino acids in adjacent 3-mer tokens. The DNA sequence may be divided similarly into overlapping 5-mer tokens, and each 5-mer may be associated with a set of three 3-mers corresponding to a particular seven amino acid zinc finger array. This tokenization process enables the machine learning model to learn context provided by the adjacent overlapping zinc finger tokens and overlapping DNA sequence tokens.

After the zinc finger arrays are generated by the machine learning model, they may be combined with a known backbone sequence to produce a complete zinc finger protein sequence. The zinc finger protein sequence may be validated using machine learning (e.g., using support vector machines) to determine if the protein sequence may be usable for wet trials.

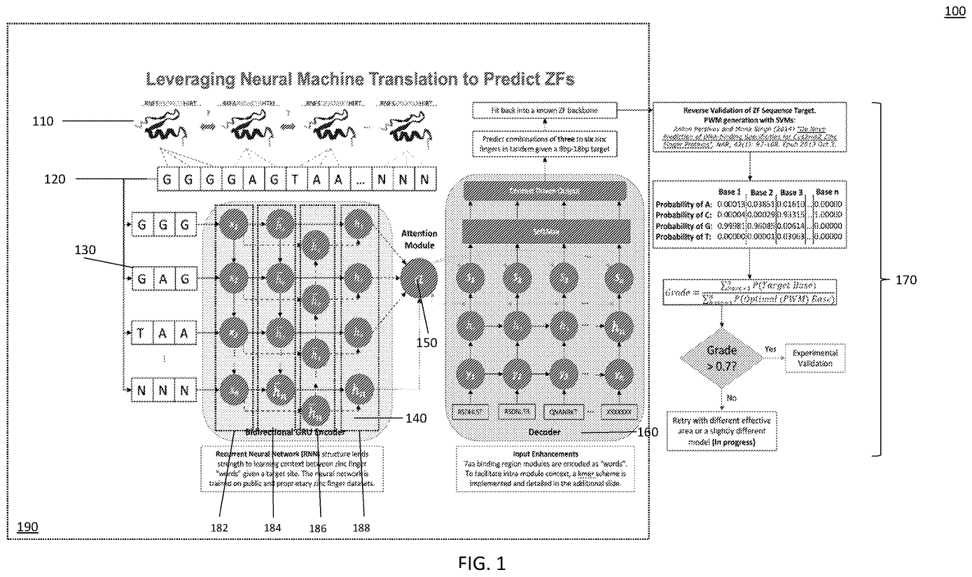

FIG. 1 illustrates a system 100 configured to generate and validate a zinc finger protein sequence, in accordance with some embodiments. The system may comprise a machine learning unit 190 for generating a plurality of zinc finger arrays corresponding to a target gene.

The machine learning unit 190 may train one or more machine learning models to generate the plurality of zinc finger arrays. Training the one or more machine learning models may comprise iteratively processing training data using the one or more machine learning models until the one or more machine learning models can accurately produce a target, or ground truth, set of zinc finger arrays when presented with a target DNA sequence.

The input data may include amino acid sequence data 110 comprising a plurality of zinc finger arrays and DNA sequence data 120 comprising binding targets 130 associated with the zinc finger arrays. The data may be collected and/or curated from publicly available repositories and/or from in-lab validated data. DNA sequence data may comprise sequences of 9-18 base pairs or more (e.g., sequences comprising a multiple of three base pairs). The amino acid sequence data 110 may comprise a sequence of at least 10, at least 20, at least 30, at least 50, at least 100, at least 150, or at least 200 amino acids. The amino acid sequence data 110 may comprise a sequence of at most 10, at most 20, at most 30, at most 50, at most 100, at most 150, or at most 200 amino acids. The amino acid sequence data 110 may comprise a sequence of between 10 and 20, between 20 and 50, between 50 and 100, between 100 and 150, or between 150 and 200 amino acids, amino acids, within which arrays of several amino acids may be interspersed. At least one array may comprise seven amino acids. At least two arrays may include the same number of amino acids. In some implementations, all zinc finger arrays have the same number of amino acids. These zinc finger arrays may be isolated from the remainder of the amino acid sequence. Using these types of sequence data allows the machine learning unit 190 to focus on a DNA region making direct contact with the zinc finger in DNA binding and may reduce the amount of input data needed to train the machine learning models.

Pre-processing the data may comprise generating a plurality of “word pairs”, with a word pair comprising a zinc finger array (e.g., of seven amino acids) and its corresponding binding site (e.g., of three base pairs (bp)) within the DNA sequence. For a seven amino acid zinc finger sequence, there may be 20⁷=1,280,000,000 words, as there are 20 amino acids in the standard genetic code. For a three base pair DNA sequence, there may be 4³=64 words.

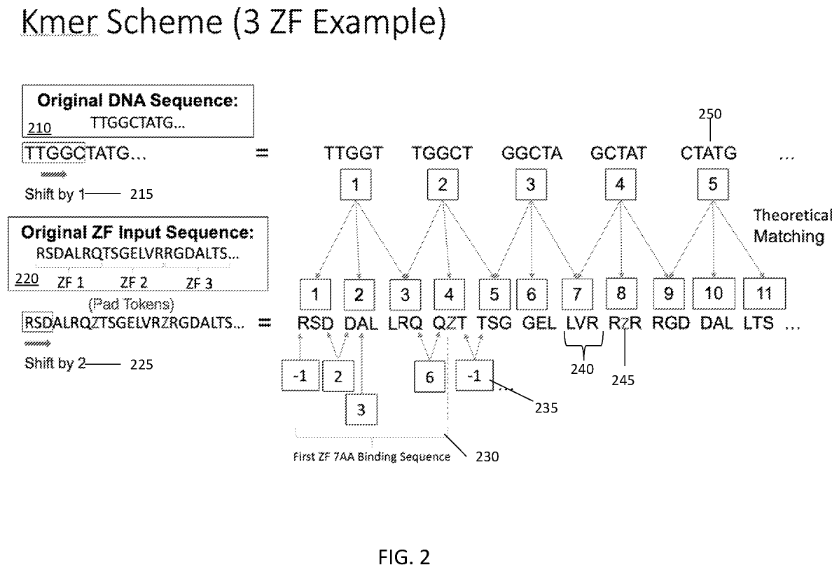

In some implementations, the input data may be further pre-processed to equalize or bring closer in magnitude the numbers of zinc finger sequence words and DNA sequence words. Pre-processing in this manner may allow the machine learning model or models to not associate too many zinc finger sequence words with a particular DNA sequence words, and may hence improve the predictive ability of the machine learning unit 190. This may be performed using a k-mer embedding scheme, as described in FIG. 2.

The machine learning unit 190 may comprise an encoder-decoder system comprising encoder 140 and decoder 160. The input data (e.g., word pairs of zinc finger arrays 110 and binding sites 120) may be used to train the encoder-decoder system. For example, training may comprise presenting DNA sequence data comprising a plurality of binding sites to the encoder 150 and presenting zinc finger array data to the decoder 160, iteratively processing the data until a convergence condition is reached (e.g., an error is minimized). The trained encoder-decoder system may generate a set of zinc finger arrays when the encoder 150 is presented with a DNA sequence comprising a plurality of binding sites. For example, a 9-18 bp DNA sequence may comprise 3-6 binding sites, from which the encoder-decoder system may predict 3-6 zinc finger arrays.

The training input sequence 130 may comprise a “sentence” of 9-18 DNA bp in length, which may include three to six binding site “words” and corresponding 3-6 zinc finger arrays.

The encoder 140 may comprise a neural network. The neural network may be a bidirectional neural network. In some implementations, the bidirectional neural network may be a bidirectional gated recurrent unit (GRU). In other implementations, the bidirectional neural network may comprise a long short-term memory (LSTM) network.

A bidirectional GRU may process an input sequence x₁ . . . xₙ from inputs 182 in both forward and backward directions. It may comprise a forward direction layer 184 for processing the input sequence in a forward direction and a backward direction layer 186 for processing the input sequence in a backward direction (e.g., the reverse of the forward direction).

In the forward direction layer 184, the GRU unit may take input (e.g., DNA sequence data from input layer 182) at each time step (x₁ . . . xₙ) and may update its hidden state (e.g., \vec{h₁} . . . \vec{hₙ}) by considering the current input and the previous hidden state. It may use gating mechanisms to control the flow of information, including an update gate and a reset gate. These gates may help the GRU decide what information to retain and what to discard.

Forward direction layer 184 may capture dependencies and patterns in the input data x₁ . . . xₙ as it progresses through the sequence. The output of forward GRU layer may be combined with the output of backward direction layer 186 to provide a more comprehensive understanding of the sequential data when working with a bidirectional GRU.

A single GRU cell of the bidirectional GRU may have the following components:
1. Update Gate (zₜ): The update gate may determine how much of the previous cell state should be preserved and how much should be updated. The value of the update gate may be determined by combining the current input (xₜ) and the previous hidden state (h₍ₜ₋₁₎) and processing them with an activation function (e.g., a sigmoid activation function).
2. Reset Gate (rₜ): The reset gate may determine which part of the previous hidden state should be forgotten and which part should be remembered. The reset gate value may be determined using the input and the previous hidden state, processed using an activation function (e.g., a sigmoid activation function).
3. Candidate Hidden State (h′ₜ): This is a candidate for the new hidden state and may be determined by applying a hyperbolic tangent (tanh) activation function to the combination of the current input and a reset-gated version of the previous hidden state (hₜ₋₁).
4. Hidden State (hₜ): The final hidden state is a combination of the previous hidden state weighted by the update gate and the candidate hidden state weighted by (1-update gate). It represents the current memory of the GRU cell. The following set of example equations shows the operations of the forward GRU at each time step (e.g., within a GRU cell). The equations may be used to generate the hidden state values for forward direction layer 184 and the hidden state values for the backward direction layer 186 from input word pairs in 182. W and U may comprise weight vectors or matrices applied to input word pairs or hidden states).

### Forward GRU Example Equations:

$$Update gate ( {z}_{t} ) : {z}_{t} = σ ( {W}_{z} * {x}_{t} + {U}_{z} * {h}_{t - 1} )$$

$$Reset Gate ( {r}_{t} ) : {r}_{t} = σ ( {W}_{r} * {x}_{t} + {U}_{r} * {h}_{t - 1} )$$

$$Candidate Hidden State ( ( {h}_{t}^{′} ) ) : ( {h}_{t}^{′} ) = tanh ( W * {x}_{t} + {r}_{t} * ( U * {h}_{t - 1} ) )$$

$$Hidden State ( {h}_{t} ) : {h}_{t} = ( 1 - {z}_{t} ) * ( {h}_{t}^{′} ) + {z}_{t} * {h}_{t - 1}$$

Backward direction layer 186 in a bidirectional gated recurrent unit (GRU) may process the inputs 182 in a reverse direction from that of forward direction layer 184.

In backward direction layer 186, the encoder (e.g., encoder 140) takes the input at each time step (e.g., x₁ . . . xₙ) and updates its hidden state (e.g., [symbol] ) by considering the current input (e.g., the input corresponding to the time step) and the previous hidden state, just like the forward direction layer 184.

Like the forward direction layer, the backward direction layer may use gating mechanisms, including an update gate and a reset gate, to control the flow of information and capture dependencies and patterns in the input data as it moves backward through the sequence.

By processing the data in both forward and backward directions, the bidirectional GRU can capture contextual information from both past and future time steps, enhancing its ability to model sequential data. The outputs of the backward direction layer 186 may be used alongside the outputs of the forward direction layer 184 to generate combined hidden states. The following is a list of example equations for the backward GRU.

### Backward GRU Example Equations:

$$Update gate ( {z}_{t}^{B} ) : {z}_{t}^{B} = σ ( {W}_{z}^{B} * {x}_{t}^{B} + {U}_{z}^{B} * {h}_{t + 1}^{B} )$$

$$Reset Gate ( {r}_{t}^{B} ) : {r}_{t}^{B} = σ ( {W}_{r}^{B} * {x}_{t}^{B} + {U}_{r}^{B} * {h}_{t + 1}^{B} )$$

$$Candidate Hidden State ( ( {h}_{t}^{′ B} ) ) : ( {h}_{t}^{′ B} ) = tanh ( {W}^{B} * {x}_{t}^{B} + {r}_{t}^{B} * ( {U}^{B} * {h}_{r + 1}^{B} ) )$$

$$Hidden State ( {h}_{t}^{B} ) : {h}_{t}^{B} = ( 1 - {z}_{t}^{B} ) * ( {h}_{t}^{′ B} ) + {z}_{t}^{B} * {h}_{t + 1}^{B}$$

In the equations for the forward GRU and backward GRU:
(xₜ) represents the input at time step t.
(hₜ) and

$$( {h}_{t}^{B} )$$

are the hidden states of the forward and backward GRU layers, respectively.
(W), (U), (W_{z}), (U_{z}), (Wᵣ), and (Uᵣ) are weight matrices associated with different gates and transformations.
σ denotes the sigmoid activation function.
tanh represents the hyperbolic tangent activation function.

Attention module 150 may allow the machine learning unit to “focus” on portions of data that may have greater predictive power for producing suitable zinc finger arrays for particular sequences of DNA base pairs. The attention module 150 may also mitigate effects of bias introduced by use of a recurrent neural network. Attention module 150 may operate on the output hidden states 188 produced by encoder 140 and the hidden states produced by decoder. Attention module 150 may transform output hidden states 188 into three sets of vectors: query, key, and value, each of which may be learned from the data and may have different dimensionality from the others. The query vector may be compared to the key vectors to compute a score for each key. Attention module 150 may perform the scoring using methods such as dot product, scaled dot product, or other measures of similarity. The scores may be processed using an activation function (e.g., a softmax function) to obtain attention weights, which may represent the importance of each key with respect to the query. The weights may be applied to the value vectors to generate an output for the decoder, reflecting the importances applied by the attention module to various portions of the input data (e.g., output hidden states 188).

Decoder 160 may be trained using an input set of zinc finger arrays and the output from attention module 150. The input set of zinc finger arrays (y₁ . . . yₙ) correspond (e.g., form word pairs with) the DNA binding sites used as input for encoder 150. Decoder 160 may generate embeddings for the input set of zinc finger arrays (y₁ . . . yₙ), and then may use these embeddings to generate hidden states (h₁ . . . hₙ) corresponding to (y₁ . . . yₙ). The hidden states (h₁ . . . hₙ) may be processed by attention module 150 and combined, also at attention module 150, with hidden states (h₁ . . . hₙ) from encoder 150 to produce attention-weighted hidden states (s₁ . . . sₙ). Decoder 160 may then process these attention-weighted hidden states with an activation function, such as a softmax function. This result may then be used to generate, for a time step, a zinc finger array associated with a DNA binding region.

The trained decoder 160 may generate a set of zinc finger arrays using the output from attention module 150. For example, the trained decoder may not receive any zinc finger arrays as input.

The decoder 160 may comprise a recurrent neural network. For example, the decoder 160 may comprise a GRU or an LSTM.

After the zinc finger arrays are generated, they may be placed into a backbone to form a complete zinc finger protein sequence with a catalytic or effector domain.

In some cases, individual amino acids of zinc finger arrays may be swapped with other amino acids to produce zinc finger protein sequences with similar effects.

Validation system 170 may be used to verify whether the generated zinc finger protein sequence corresponds to the target DNA sequence used by the machine learning unit to generate them. The zinc finger protein sequence may be processed by a support vector machine model (SVM) to determine strengths of associations of 4-bp binding sites with the zinc finger arrays of the zinc finger protein sequence. A strength of association may be determined mathematically based at least in part on the zinc finger protein's ability to bind to a 4 bp sequence. The 4-bp binding sites may each comprise DNA bases that bind to a zinc finger array at the −1, 2, 3, and 6 positions. The SVM may be trained to produce scores for any binding site combination of four base pairs (e.g., all combinations aaaa, aaat, aaag, aaac, . . . cccc), which are used to then generate a position probability matrix (PPM). From the position probability matrix, a predicted optimal DNA target is extracted and used in a base-to-base probability comparison with the intended target sequence used as input into the machine learning unit. This results in a filterable confidence metric prior to progression in wet trials.

The sequence and reverse complement of a 500 bp region before or after the transcription start site (TSS) of a target gene may be processed exhaustively to maximize the potential to find at least one high ranking zinc finger array sequence. Candidate predicted zinc finger array sequences may be subsequently transfected in vitro to target hPCSK9 and assayed experimentally with quantitative polymerase chain reaction (qPCR) on encoding RNA transfected cells.

In some implementations, the machine learning unit may use one or more natural language processing models, such as transformers, to generate the zinc finger arrays from the input DNA sequence.

FIG. 2 illustrates a k-mer embedding method 200, in accordance with some embodiments. The k-mer embedding method may pre-process the amino acid sequence data 220 and DNA sequence data 210 to generate numbers of possible zinc finger sequence words and DNA sequence words that are close in magnitude (e.g., on the order of the same power of ten). This may serve to reduce the complexity of the machine learning model, improving training and prediction.

The zinc finger arrays data and DNA sequence data may be partitioned into k-mers. The zinc finger k-mers (or “tokens”) may be of a first size and the DNA sequence data k-mers (or “tokens”) may be of a second size. The first size may or may not be equal to the second size. In some implementations, the zinc finger k-mers may have a size of three amino acids, while the DNA sequence data k-mers may have a size of five DNA base pairs. A set of three overlapping zinc finger 3-mers (overlapping, e.g., by one amino acid by shifting along the sequence with a stride length (225) of two) may comprise a single seven-amino acid zinc finger sequence array (e.g., array 230). The machine learning unit (e.g., machine learning unit 190) may also pad the zinc finger tokens with a terminal array element (e.g., 245) comprising a symbol that does not designate an amino acid. The DNA k-mers may also overlap. For example, the DNA k-mers may be 5-mers (e.g., 250) with a stride length (215) of 1. In this way, the machine learning unit (e.g., machine learning unit 190) may be able to generate 4⁵=1024 DNA sequence words and 21³=9,261 zinc finger sequence words, where 20 amino acids and one terminal element yield 21 possible choices for each “letter” of a word.

A DNA sequence k-mer (e.g., 250) may be paired with a plurality of zinc finger k-mers 240. The zinc finger k-mers of a zinc finger array sequence, due to the shifting used to generate them, overlap and thus each set of zinc finger k-mers comprises duplicate or repeated amino acids. The k-mer embedding scheme 200 may configure these overlaps to occur at binding positions of the zinc finger (e.g., amino acids corresponding to positions −1, 2, 3, and 6, where the seven amino acids of the zinc finger array are numbered by position −1, 1, 2, 3, 4, 5, and 6). A binding position may be a position of the zinc finger array that binds to one base of the DNA sequence. A position may be associated with a binding of the zinc finger with the DNA sequence at either the sense strand (oriented in the 5′ direction to 3′ direction) or the antisense strand (oriented in the opposite direction). Replicating the amino acids corresponding to the binding positions may reduce prediction errors, as adjacent zinc finger tokens must match where they share an amino acid to be successfully stitched into a seven amino acid zinc finger array.

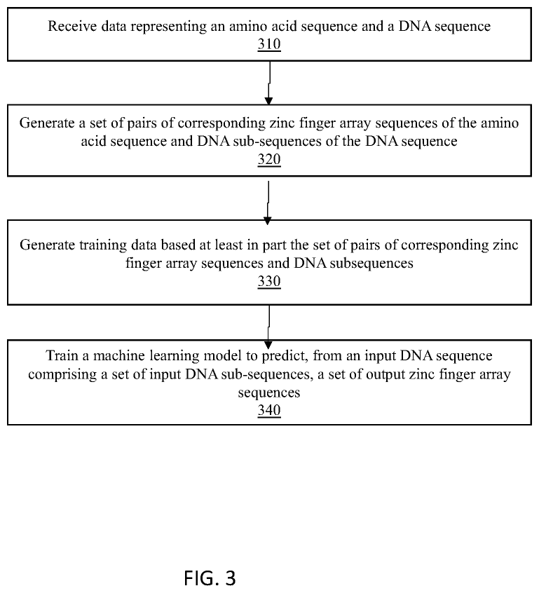

FIG. 3 illustrates a process flow diagram 300 for training a machine learning unit (e.g., machine learning unit 190), in accordance with some embodiments.

In a first operation 310, the machine learning unit receives data representing an amino acid sequence and a DNA sequence.

The machine learning unit may comprise one or more machine learning models. In some implementations, a machine learning model comprises a neural network. In some implementations, the neural network is a recurrent neural network. In some implementations, the neural network is a GRU. In some implementations, the neural network is an LSTM. In some implementations, the recurrent neural network is a bidirectional recurrent neural network. In some implementations, the bidirectional recurrent neural network is a bidirectional GRU.

The amino acid sequence may comprise a text sequence of symbols representing amino acids. The symbols may be letters. The amino acid sequence may comprise one or more zinc finger arrays. In some implementations, the zinc finger arrays comprise seven amino acids, and are represented by a sequence of seven symbols (e.g., letters) corresponding to the amino acids. The DNA sequence may correspond a sequence of DNA bases of a strand of DNA. The DNA sequence may comprise sub-sequences of symbols representing DNA bases. The DNA sequence may comprise binding regions for the zinc finger arrays. These binding regions may be three bases in length.

In a second operation 320, the machine learning unit generates a set of pairs of corresponding zinc finger array sequences of the amino acid sequence and DNA sub-sequences of the DNA sequence. The zinc finger array sequences and corresponding DNA sub-sequences may form a “word pair” that is analyzed as a package by the machine learning unit. In some implementations, a machine learning unit may analyze “sentences” comprising three to six word pairs.

In a third operation 330, the machine learning unit generates training data based at least in part the set of word pairs of corresponding zinc finger array sequences and DNA subsequences. The training data may comprise embeddings, representations, or vectorizations of the word pairs. In some implementations, generating the training data comprises pre-processing.

In some implementations, the machine learning unit may use a k-mer embedding scheme to generate the training data from the amino acid sequence and DNA sequence. The k-mer embedding scheme, as referred to by FIG. 2, may be configured to generate a total possible number of “zinc finger words” that is relatively close in magnitude (e.g., on the order of the same power of ten) to the total possible number of “DNA binding site words”, of which the word pairs are comprised.

The k-mer embedding scheme may generate DNA sequence tokens and zinc finger array sequence tokens. The set of DNA sequence tokens may be generated by selecting k consecutive bases, and then shifting and selecting a next set of k consecutive bases. For example, a first DNA sequence token may comprise five bases, while a second DNA sequence token may be shifted with a stride length of 1, so that there is a four-base overlap between the first and second DNA sequence tokens. The shift may be to the right. The set of zinc finger sequence tokens may be generated by selecting k consecutive amino acids, and then shifting and selecting a next set of k consecutive amino acids. For example, a first zinc finger token may comprise three amino acids, while a second zinc finger token may be shifted with a stride length of 2, so that there is a one-amino acid overlap between the first and second zinc finger tokens. The shift may be to the right. In this example, three zinc finger 3-mers would form a single seven amino acid zinc finger array, with duplicate or repeated amino acids. The terminal 3-mer may be padded with an additional array element that does not represent an amino acid.

In a fourth operation 340, the machine learning unit trains a machine learning model to predict, from an input DNA sequence comprising a set of input DNA sub-sequences, a set of output zinc finger array sequences. The machine learning model may comprise an encoder-decoder system. The encoder may be configured to process the word pairs to learn context between zinc finger “words” given a target site. The encoder may produce a plurality of hidden states corresponding to a plurality of word pairs. The machine learning unit may comprise an attention mechanism to weight the hidden states to account for bias (e.g., recency bias produced by a recurrent neural network) and improve the predictive power of the machine learning model. The decoder may process the attention-weighted hidden states to generate an output set of zinc finger arrays. On successive iterations of training, the output set of zinc finger arrays may be fed back into the decoder to produce previous output hidden states which may be combined or processed with the attention-weighted hidden states for the subsequent iterations.

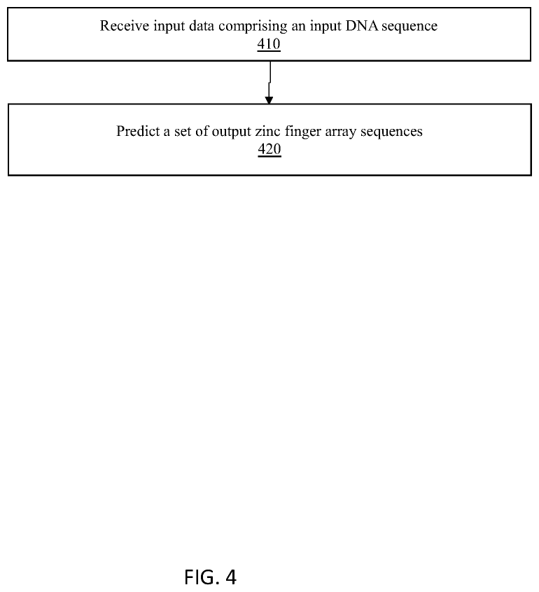

FIG. 4 illustrates a process flow diagram 400 for using a machine learning unit (e.g., machine learning unit 190) to generate a set of output zinc finger array sequences, in accordance with some embodiments.

In a first operation 410, the machine learning unit receives input data comprising an input DNA sequence. The input data may comprise a target DNA sequence. The target DNA sequence may comprise, for example, 9-18 base pairs.

In a second operation 420, the machine learning unit predicts a set of output zinc finger array sequences from the DNA sequence. The machine learning unit may predict, for example, three to six zinc finger arrays given a 9-18 base pair target. The zinc finger arrays may be fit into a backbone sequence to generate a complete zinc finger protein sequence. A zinc finger backbone may comprise an alpha helix and a beta sheet stabilized by a zinc ion. The complete zinc finger protein sequence may be validated to demonstrate that it corresponds to the target sequence. Validation may be performed using, for example, an SVM-based method.

The following sections describe systems and methods for validating the zinc finger protein sequences which may be produced by the machine learning methods herein. The following sections should not be construed to limit any of the preceding disclosure.

### In Vitro Validation

### In Vitro Transcription

DNA templates for generating RNA products were created by polymerase chain reaction (PCR) amplification from plasmids or gBlock gene fragments and purified using a PCR purification kit. Plasmids were then generated with these templates containing a T7 promoter followed by 5′ ribozyme sequence, a 5′ ligation sequence, an IRES sequence linked to the zinc finger array (ZFA) protein and respective activation domains, a 3′ UTR sequence, a 3′ ligation sequence, a 165 bp poly-A stretch, a 3′ ribozyme sequence, and lastly, a poly-T stretch to terminate transcription. All experiments utilized m6A modified RNA, for which an RNA synthesis kit was used, where 5% of adenosine triphosphate (ATP) was substituted with N6-Methyladenosine-5′-.

### In Vitro Gene Modulation Experiments

To assess modulation of target genes, HEK293T cells (CXCR4, ASCL1) and HeLa (PCSK9) cells were transfected with ribonucleic acid (RNA) encoding for a ZFA-KRAB and RNA was isolated at 48 hours after transfection. cDNA synthesis was performed and subsequently qPCR to assess the amount of gene expression relative to GAPDH.

In epigenetic silencing experiments, 0 hours marked transfection of each RNA encoding for a 3A3L-ZFA-KRAB protein, and the “mix” condition representing a mix of all three zinc finger arrays in equal proportion, the sum of which totaled the same final transfected moles of RNA of the individual conditions. Cells were seeded 24 hours prior, achieving a 40% confluency at transfection and passaged once approximately 95% confluent, every two to four days, for a total of 14 days. Each time point represents average qPCR data retrieved from 75% of cells collected from each triplicate well for each condition, with 25% passaged or removed regarding the last collection. Both conditions were collected in the same manner concurrently. cDNA synthesis was performed and subsequently qPCR to assess the amount of gene expression relative to GAPDH. With comparison, it appears possible to attach a domain to both —C and —N terminals of the zinc finger array at once, and to facilitate an inherited repressive effect with a chromatin remodeling epigenetic domain.

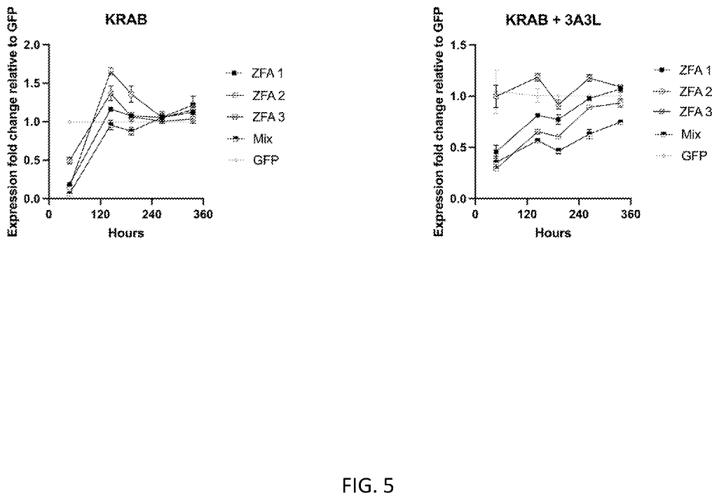

FIG. 5 illustrates instanced repression and inheritable silencing by zinc finger arrays bound to KRAB and KRAB+3A3L respectively.

### Backbone Modifications

All six zinc finger predictions are combined with a backbone, collected from previously validated experiments and research. Since zinc finger backbones such as Zif268 and SP1 only contain three zinc finger domains, fusions capable of six finger domains are attained by linking two units together or with synthetic linkers between each finger derived experimentally to produce a flexible construct retaining zinc finger binding effectiveness. Further, modifications to specific amino acids in the backbone, (e.g., arginine slightly upstream of each seven amino acid DNA contact region being changed to an Alanine or a Glutamine), have been shown to improve holistic zinc finger specificity to the intended target site. Additional backbone modifications of these forms have also shown improvements to specificity, as described by works in the field, demonstrating a wide variety of changes to backbone and single amino acid substitutions with the DNA contacting amino acids to improve specificity of a zinc finger array. Other modifications specific to positions in common linker sequences such as TGEKP have also been highlighted to improve affinity to DNA sequences. As shown by Table 1. In the following section, Backbone Options, the zinc finger predictions are marked by seven Xs to demote the seven amino acids which are predicted. Furthermore, the examples annotated illustrate six regions for ZF-DNA contact, wherein ZF1 to ZF6 are inserted from −N to −C terminal of the amino acids.

TABLE 1
Modifications and Integration of Backbone for
Sequence Specific ZFAs

| | |
|---|---|
| DNA Contact Region of | XXXXXX |
| a zinc finger (7 | |
| amino acid) | |
| Amino Acid Substitution | Arg: R > Ala: A or |
| for Specificity | Glu: G, or any of the |
| | remaining AAs. |
| Linker Region for | TGEKP |
| Modification | |

### Backbone Options

Some backbones capable of use with the predicted 7 aa zinc finger sequence include, but are not limited to including:

Backbone Positional Diagram of Insertions
(Backbone 1)
MAPKKKRKVGIHGVPAAMAERPFQCRICMRNFS(ZF1)HIRTHTGEKPFA
CDICGRKFA(ZF2)HTKIHTGSQKPFQCRICMRNFS(ZF3)HIRTHTGEK
PFACDICGRKFA(ZF4)HTKIHTGSQKPFQCRICMRNFS(ZF5)HIRTHT
GEKPFACDICGRKFA(ZF6)HTKIHLRQKDAARGS
Backbone 1
MAPKKKRKVGIHGVPAAMAERPFQCRICMRNFSXXXXXXXHIRTHTGEKP
FACDICGRKFAXXXXXXXHTKIHTGSQKPFQCRICMRNFSXXXXXXXHIR
THTGEKPFACDICGRKFAXXXXXXXHTKIHTGSQKPFQCRICMRNFSXXX
XXXXHIRTHTGEKPFACDICGRKFAXXXXXXXHTKIHLRQKDAARGS
Backbone 2 - Aart²⁵
ISEFGSSSSVAQAALEPGEKPYACPECGKSFSRXXXXXXXHQRTHTGEKP
YKCPECGKSFSXXXXXXXHQRTHTGEKPYKCPECGKSFSXXXXXXXHQRT
HTGEKPYKCPECGKSFSXXXXXXXHQRTHTGEKPYKCPECGKSFSXXXXX
XXHQRTHTGEKPYKCPECGKSFSXXXXXXXHQRTHTGKKTS
Backbone 3 - SP1¹
VPIPGKKKQHICHIQGCGKVYGXXXXXXXHLRWHTGERPFMCTWSYCGKR
FTXXXXXXXHKRTHTGEKKFACPECPKRFMXXXXXXXHIKTHQNKKDGGG
SGKKKQHICHIQGCGKVYGXXXXXXXHLRWHTGERPFMCTWSYCGKRFTX
XXXXXXHKRTHTGEKKFACPECPKRFMXXXXXXXHIKTHQNKKGGS

### Validated Zinc Finger Arrays and Linked Domains

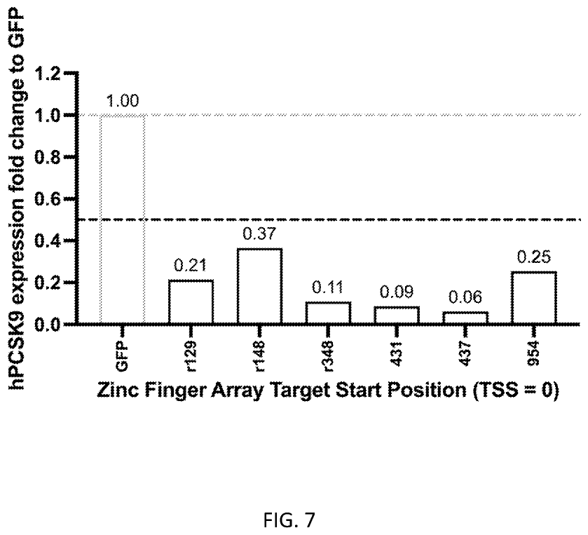

In vitro validated zinc finger domains are shown in Tables 3-5 for which the corresponding qPCR data is shown in FIGS. 7-9. A requirement of above 50% repression was required for a ZFA-KRAB to be deemed as successfully targeting the gene of interest. It is also noted that while six finger polydactyl zinc fingers were chosen to be further evaluated, generation of 2-6 zinc finger arrays targeting corresponding 6-18 base pairs (bp) of target DNA with increments of three bp are technically possible and were observed. Each zinc finger array is annotated via the start or 5′ end of the proposed DNA target in the gene of interest where the transcription start site (TSS) represents zero of the sense strand as shown in FIGS. 7-9. The prefix “r” is also included as a representation that the zinc finger array is targeting the antisense strand while those without are targeting the sense strand. However, those on the antisense strand retain the positional number of the sense strand but marking the position of the 5′ end of the intended target on the antisense strand. For example, a six finger zinc finger array corresponding with a DNA target length of 18 bp, targeting the first 18 bp from the TSS on the sense strand would have a position index of “0”, while a zinc finger array targeting the same, complementary, region on the antisense strand would have a position index of “r18.”

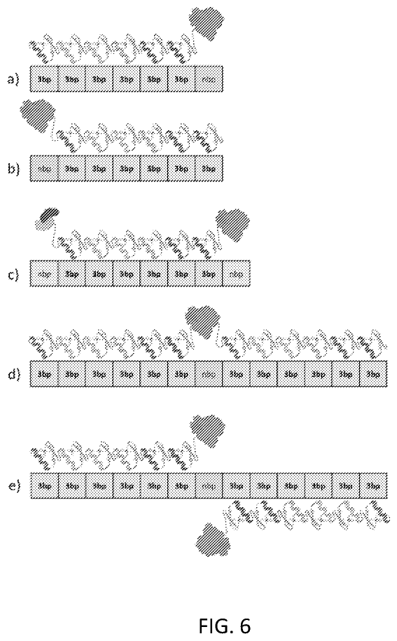

The zinc finger arrays described here are capable of attachment with domains including combinations thereof but not limited to the ones described in Table 1. Furthermore, attachment of these domains can be performed on either the —C terminal or —N terminal domain, or both, as in the 3A3L-KRAB inherited silencing experiments where the 3A3L fusion protein is bound on the —N terminal while the KRAB domain is bound on the —C terminal domain to achieve the desired effect. For targeting strategies, conformations including all combinations thereof but not limited to those shown and described in FIG. 6 can be employed to best deliver the zinc finger array and domain to the desired genetic target. Peptide linkers are also included between zinc finger array and attached domains, in widely varying forms commonly characterized in the field.

TABLE 2
Some Proposed Domains for Fusion with Predicted ZFAs.
−C or −N Terminal Fusion Domains for Sequence Specific Effect

| | | Other |
|---|---|---|
| | | (Base Editing, Chromatin |
| Activators | Repressors | Remodeling) |
| VP16 | KRAB | 3A3L |
| VP64 | ZIM2/3 | MSSSI |
| VPR | rTA | PRMI |
| KLF4/6 | | PRMII |
| SOX2 | | FOKI |
| NANOG | | |
| MYC | | |
| OC | | |
| T4 | | |
| LIN28 | | |

FIG. 6 illustrates zinc finger array and domain DNA binding conformations, in accordance with some embodiments. a) illustrates C-terminal fusion, b) illustrates N-terminal fusion, and c) both N-terminal and C-terminal fusion of a single zinc finger array with a protein domain. d) illustrates a C-terminal fusion of one zinc finger array and an N-terminal fusion of another zinc finger array to a shared single protein domain. e) illustrates two C-terminal or N-terminal fusions of protein domains with their respective zinc finger arrays, binding on nearby regions of opposing complementary DNA strands.

TABLE 3
hPCSK9 Zinc Finger Regions for Targeted Repression in HeLa cells
hPCSK9 Repression

| Position | ZF1 | ZF2 | ZF3 | ZF4 | ZF5 | ZF6 |
|---|---|---|---|---|---|---|
| r96 | RSDSLSR | RNASLAT | DRSDLSR | DRSDLSR | DRSALSR | QSGTLAR |
| r129 | RSSDLTE | RSADLSR | QSSDLSR | HRSTLSR | QSSDLSR | QSSTLSR |
| r148 | QSSDLSR | QSGHLSR | RSDVLSE | RNQHRKT | YPKDLSK | QSGNLAR |
| r348 | RSDSLTR | QSGDLTR | QSGDLTR | QSGDLTR | QSGDLTR | QSGDLTR |
| 431 | ERGTLTR | RSDALAR | DRDSLTR | DRSNLAR | RSDNLAR | RSDNLTR |
| 437 | QSGSLTR | QSGNLAR | ERRDLSR | RSDALPM | DRSDLSR | DRSNLTR |
| 954 | RSDNLSR | RSADLSR | QSGDLTR | RRDHLTT | QSDVLSR | QSATLSK |

FIG. 7 illustrates successful six finger ZFA-KRAB Fusions for >100% hPCSK9 activation.

TABLE 4
CXCR4 Zinc Finger Regions for Targeted Repression in HEK293T cells
CXCR4 Repression

| Position | ZF1 | ZF2 | ZF3 | ZF4 | ZF5 | ZF6 |
|---|---|---|---|---|---|---|
| 7 | RSDNLSR | DRGTLAR | DRSNLSR | QSSNLAR | RSDSLSR | DRSNRKT |
| 10 | QSGTRKT | RSDNLAR | DRSDLTR | DRSNLAK | QSSHLAR | RSDALAR |
| 16 | QSSNLVR | RSDKLSR | QSSALSR | RSDNLAR | DRSDLSR | DRSNLSR |

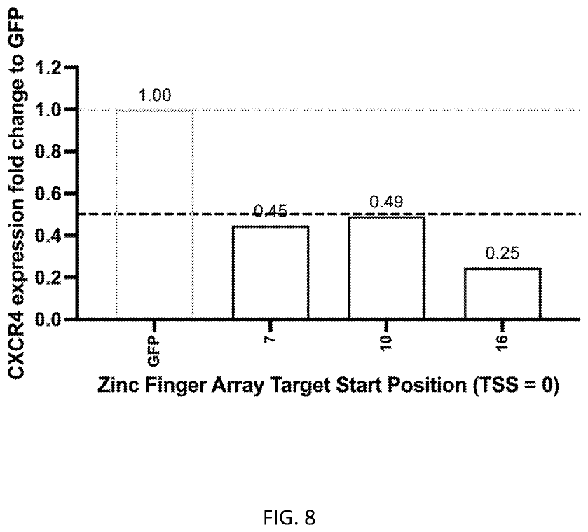

FIG. 8 illustrates successful six finger ZFA-KRAB fusions for >50% CXCR4 repression.

TABLE 5
ASCL1 Zinc Finger Regions for Targeted Activation in HEK293T cells
ASCL1 Activation

| Position | ZF1 | ZF2 | ZF3 | ZF4 | ZF5 | ZF6 |
|---|---|---|---|---|---|---|
| r294 | ERGTLAR | QSSDLRR | QSSDLSR | RSDHLRR | RSDHLSR | RSDDRKT |
| 313 | RSDNLAR | RSDNLAR | RSAHLSR | RSDHLSR | RSDHLSN | RSDHRKT |
| r324 | DRSHLSR | RSDHLSR | RSDALAR | RSDHLSR | RSDNLSA | RKHDRIT |
| 327 | RSDDLAR | RSDHLSR | RSDNLAR | RSDHLAR | RSDNLAR | QSGHLSR |
| 347 | RSDHLSA | RSAHLTN | RSDHLSR | RSDALAR | QSGHLAR | RSDHLTT |
| 379 | QSSDLRR | RSDHLSR | RSDSLLR | QSGHLTR | RSDHLSR | RSDHLSR |
| 388 | QSSDLTR | DRKSLAR | QSGDLTR | QSGHLSR | RSDHLSR | RSDALAR |
| 420 | RSDDLTR | RSDNLAR | DRSHLSR | RSDHLSR | RSDHLSR | RNDHRKR |
| 423 | RSDHLSR | RSADLRR | RSDNLAR | DRSHLAR | RSDHLSR | RSDHRKT |

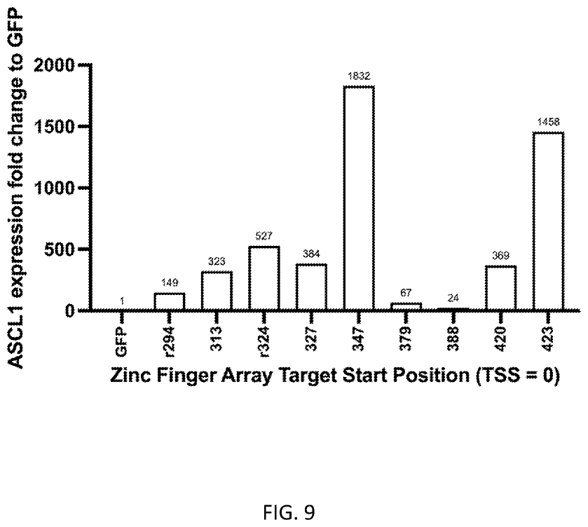

FIG. 9 illustrates successful six finger ZFA-KRAB fusions for >50% ASCL1 repression.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:

### Example 1

A training method, comprising: receiving data representing an amino acid sequence and a DNA sequence; based on the received data, generating a set of pairs of corresponding zinc finger array sequences of the amino acid sequence and DNA sub-sequences of the DNA sequence, wherein a DNA sub-sequence comprises a binding site for a corresponding zinc finger array sequence; generating training data based at least in part the set of pairs of corresponding zinc finger array sequences and DNA subsequences; training, using the training data, a machine learning model to predict, from an input DNA sequence comprising a set of input DNA sub-sequences, a set of output zinc finger array sequences, an output zinc finger array sequence of the set of output zinc finger array sequences corresponding to an input DNA sub-sequence of the set of input DNA sub-sequences.

### Example 2

The method of Example 1, further comprising, prior to training the machine learning model, pre-processing the zinc finger array sequences and the DNA sub-sequences, the pre-processing comprising: padding each of the zinc finger array sequences with a terminal array element, wherein the terminal array element does not comprise a symbol used to designate an amino acid; dividing a zinc finger array sequence of the zinc finger array sequences into a plurality of zinc finger sub-sequences, wherein a first zinc finger sub-sequence and a second zinc finger sub-sequence share an array element, wherein the array element is not the terminal array element; and generating an association of the plurality of zinc finger sub-sequences with a DNA sub-sequence, wherein the association is configured to comprise a repetition of at least one shared array element of the zinc finger array sequence, wherein the shared array element corresponds to a DNA binding amino acid.

### Example 3

The method of Example 2, wherein a position of the DNA binding amino acid is −1, 2, 3, or 6.

### Example 4

The method of any of Examples 2-3, wherein a first DNA sub-sequence of the DNA sub-sequences is a 5-mer and a second DNA sub-sequence of the DNA sub-sequences is a 5-mer.

### Example 5

The method of Example 4, wherein the first DNA sub-sequence is a first set of consecutive bases of the DNA sequence and the second DNA sub sequence is a second set of consecutive bases of the DNA sequence, wherein the second set is determined at least in part by shifting the first set.

### Example 6

The method of Example 5, wherein the shifting is by one base of the DNA sequence.

### Example 7

The method of any of Examples 5-6, wherein the shifting is rightward.

### Example 8

The method of any of Examples 2-7, wherein the first zinc finger sub-sequence is a 3-mer and the second zinc finger sub-sequence is a 3-mer.

### Example 9

The method of Example 8, wherein the first zinc finger sub-sequence corresponds to a first set of amino acids of the zinc finger array sequence, wherein the second zinc finger sub-sequence corresponds to a second set of amino acids of the zinc finger array sequence, wherein the second zinc finger sub-sequence is determined by shifting the first zinc finger sub-sequence.

### Example 10

The method of Example 9, wherein the shifting is rightward.

### Example 11

The method of any of Examples 9-10, wherein the shifting is by two amino acids of the zinc finger array sequence.

### Example 12

The method of any of Examples 1-11, wherein the trained machine learning model comprises a neural network.

### Example 13

The method of Example 12, wherein the neural network is a bidirectional neural network.

### Example 14

The method of Example 13, wherein the bidirectional neural network comprises a gated recurrent unit (GRU).

### Example 15

A prediction method, comprising: receiving input data comprising an input DNA sequence; and predicting, using a trained machine learning model, a set of output zinc finger array sequences, an output zinc finger array sequence corresponding to a DNA sub-sequence of the input DNA sequence, wherein the DNA sub-sequence comprises a binding site for the corresponding output zinc finger array sequence.

### Example 16

The Example of claim 15, wherein the trained machine learning model comprises a neural network.

### Example 17

The Example of claim 16, wherein the neural network is a bidirectional neural network.

### Example 18

The Example of claim 17, wherein the bidirectional neural network comprises a gated recurrent unit (GRU).

### Example 19

The method of any of Examples 15-17, further comprising inserting the set of output zinc finger array sequences into a zinc finger backbone.

### Example 20

The method of Example 19, further comprising generating a confidence metric at least in part by processing the zinc finger backbone comprising the set of output zinc finger array sequences with a second machine learning model.

### Example 21

The method of Example 20, wherein the second machine learning model comprises a support vector machine model.

### Example 22

The method of any of Examples 15-21, wherein a zinc finger array sequence comprises a 7-mer amino acid sequence.

### Example 23

The method of any of Examples 15-22, wherein a DNA sub-sequence comprises a 3-mer DNA base sequence.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

1. A training method, comprising:
receiving data representing an amino acid sequence and a DNA sequence;
based on the received data, generating a set of pairs of corresponding zinc finger array sequences of the amino acid sequence and DNA sub-sequences of the DNA sequence, wherein a DNA sub-sequence comprises a binding site for a corresponding zinc finger array sequence;
generating training data based at least in part the set of pairs of corresponding zinc finger array sequences and DNA subsequences; and
training, using the training data, a machine learning model to predict, from an input DNA sequence comprising a set of input DNA sub-sequences, a set of output zinc finger array sequences, an output zinc finger array sequence of the set of output zinc finger array sequences corresponding to an input DNA sub-sequence of the set of input DNA sub-sequences.

2. The method of claim 1, further comprising, prior to training the machine learning model, pre-processing the zinc finger array sequences and the DNA sub-sequences, the pre-processing comprising:
padding each of the zinc finger array sequences with a terminal array element, wherein the terminal array element does not comprise a symbol used to designate an amino acid;
dividing a zinc finger array sequence of the zinc finger array sequences into a plurality of zinc finger sub-sequences, wherein a first zinc finger sub-sequence and a second zinc finger sub-sequence share an array element, wherein the array element is not the terminal array element; and
generating an association of the plurality of zinc finger sub-sequences with a DNA sub-sequence, wherein the association is configured to comprise a repetition of at least one shared array element of the zinc finger array sequence, wherein the shared array element corresponds to a DNA binding amino acid.

3. The method of claim 2, wherein a position of the DNA binding amino acid is −1, 2, 3, or 6.

4. The method of claim 2, wherein a first DNA sub-sequence of the DNA sub-sequences is a 5-mer and a second DNA sub-sequence of the DNA sub-sequences is a 5-mer.

5. The method of claim 4, wherein the first DNA sub-sequence is a first set of consecutive bases of the DNA sequence and the second DNA sub-sequence is a second set of consecutive bases of the DNA sequence, wherein the second set is determined at least in part by shifting the first set.

6. The method of claim 5, wherein the shifting is by one base of the DNA sequence.

7. The method of claim 5, wherein the shifting is rightward.

8. The method of claim 2, wherein the first zinc finger sub-sequence is a 3-mer and the second zinc finger sub-sequence is a 3-mer.

9. The method of claim 8, wherein the first zinc finger sub-sequence corresponds to a first set of amino acids of the zinc finger array sequence, wherein the second zinc finger sub-sequence corresponds to a second set of amino acids of the zinc finger array sequence, wherein the second zinc finger sub-sequence is determined by shifting the first zinc finger sub-sequence.

10. The method of claim 9, wherein the shifting is rightward.

11. The method of claim 9, wherein the shifting is by two amino acids of the zinc finger array sequence.

12. The method of claim 1, wherein the trained machine learning model comprises a neural network.

13. The method of claim 12, wherein the neural network is a bidirectional neural network.

14. The method of claim 13, wherein the bidirectional neural network comprises a gated recurrent unit (GRU).

15. A prediction method, comprising:
receiving input data comprising an input DNA sequence; and
predicting, using a trained machine learning model comprising a neural network, a set of output zinc finger array sequences, an output zinc finger array sequence corresponding to a DNA sub-sequence of the input DNA sequence, wherein the DNA sub-sequence comprises a binding site for the corresponding output zinc finger array sequence.

16. (canceled)

17. The method of claim 15, wherein the neural network is a bidirectional neural network and comprises a gated recurrent unit (GRU).

18. (canceled)

19. The method of claim 15, further comprising inserting the set of output zinc finger array sequences into a zinc finger backbone.

20. The method of claim 19, further comprising generating a confidence metric at least in part by processing the zinc finger backbone comprising the set of output zinc finger array sequences with a second machine learning model, wherein the second machine learning model comprises a support vector machine model.

21. (canceled)

22. The method of claim 15, wherein a zinc finger array sequence comprises a 7-mer amino acid sequence.

23. The method of claim 15, wherein a DNA sub-sequence comprises a 3-mer DNA base sequence.
